(19) United States
(12) Patent Application Publication
(10) Pub. No.: US 2005/0143473 A1
(43) Pub. Date: Jun. 30, 2005

(54) Methods for treating allergy

(76) Inventors: Gary Kwan Po Wong, Kowloon (HK); Francis Chi, Kowloon (HK)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/015,362

(22) Filed: Dec. 17, 2004

Related U.S. Application Data
(60) Provisional application No. 60/531,048, filed on Dec. 19, 2003.

(51) Int. Cl. A61K 31/13

(52) U.S. Cl. 514/665

(57) ABSTRACT

The subject invention provides materials and methods for treating allergy in animals. In a preferred embodiment, the invention provides methods for alleviating an animal's hypersensitive response to allergens, including, without limitation, migraine, asthma, allergic rhinitis, digestive disturbances, coeliac disease, conjunctivitis, urticaria, eczema, drowsiness, hyperactivity (in children), tinnitus, recurrent sinusitis and ear infections. Specifically exemplified herein is the use of a cysteamine compound to reduce the hypersensitive response in animals to allergens. In a preferred embodiment, oral administration of cysteamine hydrochloride to an animal diagnosed with an allergy can substantially reduce the animal's reaction to the allergen.

### CROSS-REFERENCE TO A RELATED APPLICATION

This application claims the benefit of U.S. provisional application Ser. No. 60/531,048, filed Dec. 19, 2003.

### BACKGROUND OF THE INVENTION

Allergies pose a serious public health problem, both in the United States and worldwide. As the 6^{th} leading cause of chronic disease in the United States, allergies are costing the health care system $18 billion annually. Centers for Disease Control. Fast Stats A-Z, Vital and Health Statistics, Series 10, No. 200, Table 57 (1996). Pollen allergy (allergic rhinitis or hay fever) affects about 10-15% of the population, generating an estimated $1.8 billion of direct and indirect expenses in the United States in 1990 alone (Fact Sheet, National Institute of Allergy and Infectious Diseases; McMenamin, Annals of Allergy 73: 35, (1994). More serious than the economic costs associated with pollen and other allergens (i.e., molds, dust mites, animal danders) is the risk of anaphylactic reaction observed with allergens such as food allergens, insect venoms, drugs, and latex.

Allergic reactions occur when an individual's immune system is hypersensitive (i.e., overreacts or reacts inappropriately) to an encountered antigen. The hypersensitive or allergic immune system misidentifies an otherwise innocuous substance (antigen) as harmful, and then attacks the antigen with a ferocity far greater than required. Specifically, the antigen is taken up by antigen presenting cells (i.e., macrophages or dendritic cells) that degrade the antigen and then display antigen fragments to T-lymphocytes (T-cells). The activated T-cells respond by secreting a collection of cytokines that affect other cells of the immune system.

The profile of cytokines secreted by responding T-cells determines whether subsequent exposures to the particular antigen will induce allergic reactions. When T-cells respond by secreting interleukin-4 (IL-4), the effect is to stimulate the maturation of B-lymphocytes (B-cells) that produce IgE antibodies specific for the antigen. These antigen-specific IgE antibodies then attach to specific receptors on the surface of mast cells and basophils. Both mast cells and basophils contain histamine and other allergy mediators. Thus, IgE antibody attachment to either mast cells or basophils acts as a trigger to initiate a rapid reaction to subsequent exposures to the antigen.

It has been hypothesized that clinical symptoms produced in the course of allergic reaction are the result of an early specific immune response and a late inflammatory reaction. For example, with inhaled allergens (i.e., pollens, mite dust), Ige receptors to either mast cells or basophils are stimulated, which in turn release histamine and cytokines. This early phase lasts for about 30 minutes. The cytokines released from mast cells and basophils then mediate the late phase by recruiting inflammatory cells into the nasal and upper respiratory tract passages (Serafin, WE, In Goodman and Gillmans “The Pharmacological Basis of Therapeutics”, Hardmen, Ja; Limbird, L,E eds, Mc Graw-Hill, N.Y., 1996, 659-682). The influx of eosinophils, macrophages, lymphocytes, neutrophils and platelets starts the vicious inflammatory cycle. This late phase lasting for 8-48 hours amplifies the initial immune response, which in turn triggers the release of more inflammatory cells (Townley R G and Okada, C, Annals of Allergy, 68, 1991, 190-196).

In line with this hypothesis, nonsteroidal anti-inflammatory medications (NSAIDs) have been proposed for use in treating allergic conditions. Unfortunately, NSAIDs do not generally provide relief from common physical symptoms (i.e., runny nose and eyes, rashes, difficulty breathing) associated with allergic response. Moreover, NSAIDs can cause some serious side effects including stomach ulcers, increased tendency to bleed, or even induce an allergic reaction (i.e., rashes, wheezing, and throat swelling).

Another approach to treating allergies is antigen immunotherapy, which attempts to “vaccinate” a sensitive individual against a particular allergen by periodically injecting or treating the individual with a crude suspension of the raw allergen. The goal is to modulate the allergic response mounted in the individual through controlled administration of known amounts of antigen. If the therapy is successful, the individual's allergic response is diminished, or can even disappear. However, the therapy can require several rounds of vaccination, over an extended time period (3-5 years), and very often does not produce the desired results. Moreover, certain individuals suffer anaphylactic reactions to the vaccines, despite their intentional, controlled administration.

The administration of histamine antagonists is also commonly used to treat allergic symptoms. For example, antihistamines such as the second generation H₁-blockers are used for the treatment of allergic conditions, including seasonal rhinitis and allergic dermatitis (Gong, H, Tashkin, D. P, Dauphinee, B et al., J. Allergy. Clin. Immunol., 85, 1990, 632-641). These drugs are widely available in over-the-counter formulations, but unfortunately they merely mask the symptoms of the allergic response rather than providing any type of permanent cure or protection against recurrence.

Efforts are underway to develop more specific treatments for allergy and asthma (see, for example, Fahy et al., Am. J. Respir. Crit. Care Med. 155: 1828, 1997; Boulet et al., Am. J. Respir. Crit. Care Med. 155: 1835, 1997; Kung et al., Am. J. Respir. Cell Mol. Bio. 12: 360, 1995; Mauser et al., Am. J. Respir. Crit. Care Med. 152: 467, 1995; Holgate et al., J. Allergy Clin. Immunol. 98: 1, 1996). Also, non-traditional treatments for these maladies are being explored. However, there remains a need for the development of improved allergy therapies.

### BRIEF SUMMARY OF THE INVENTION

The subject invention provides methods for treating allergies. In a preferred embodiment, the invention provides methods for the treatment and/or prevention of allergic symptoms as well as the prevention or delay in development of an allergic response to an antigen.

Specifically exemplified herein is the use of a cysteamine compound to reduce and/or eliminate the severity, intensity, and/or duration of at least some symptoms associated with an allergic reaction. For example, undesirable and unpleasant symptoms such as sneezing, watery eyes, and wheezing, which are commonly associated with an allergic reaction, can be reduced through consumption, according to the subject invention, of cysteamine hydrochloride.

Further advantages of the subject invention include treatment and/or prevention of development of allergies. In accordance with the subject invention, administration of a cysteamine compound to an allergic or sensitized patient can alter the immune response so that an allergic response does not develop, or develops to a lesser extent than would be observed in the absence of cysteamine.

### BRIEF DESCRIPTION OF DRAWINGS

FIG. 1 shows a metabolic pathway of cysteamine.

FIG. 2 shows cysteamine as a constituent of co-enzyme A.

### DETAILED DISCLOSURE OF THE INVENTION

The subject invention provides methods for treating allergies in patients. In preferred embodiments, the invention provides methods for treating and/or preventing the development of allergies as well as the symptoms associated with an allergic reaction.

As used herein, the term “allergic reaction” refers to a clinical response by a patient to an antigen. Symptoms of allergic reactions can affect the respiratory (i.e., coughing, laryngeal edema, shortness of breath, wheezing, rhinorrhea, watery/itching eyes and nose); cutaneous (i.e., urticaria, angioedema, pruritus); gastrointestinal (i.e., digestive disturbances such as vomiting, diarrhea, and abdominal pain); and/or cardiovascular (i.e., palpitations) systems. Additional symptoms of allergic reactions include, without limitation, migraine, asthma, allergic rhinitis, coeliac disease, conjunctivitis, eczema, drowsiness, hyperactivity (in children), tinnitus, recurrent sinusitis and ear infections. In certain cases, severe allergic reactions can affect the entire body (i.e., anaphylaxis), which can lead to death.

An “antigen,” as used herein, includes any compound or composition that elicits an immune response and/or that binds to a T-cell receptor (i.e., when presented by a major histocompatibility complex (MHC) molecule) or to an antibody produced by a B-cell. It is well known in the art that an antigen may be a collection of different chemical compounds or a single compound.

The term “patient,” as used herein, describes an organism, including mammals, to which treatment with the compositions according to the present invention is provided. Mammalian species that benefit from the disclosed methods of treatment include, and are not limited to, apes, chimpanzees, orangutans, humans, monkeys; and domesticated animals (i.e., pets) such as dogs, cats, mice, rats, guinea pigs, and hamsters.

An “allergic patient,” as used herein, refers to a patient with sensitivities to particular antigens or allergens as exhibited by eliciting an IgE response in an individual sufficient to cause a measurable allergic reaction and/or by eliciting the signs and symptoms of an allergic reaction, whether or not such a reaction includes a detectable IgE response. An allergic patient includes those patients who have a reaction to relatively innocuous antigen, as compared to those patients that do not have a similar reaction upon exposure to an identical antigen.

As used herein, the term “sensitized patient,” refers to a patient who has been exposed to a given antigen and has mounted an immune response to that antigen that results in the display of one or more allergic symptoms when the patient is exposed to the antigen.

“Concurrent administration” and “concurrently administering,” as used herein, includes administering a compound or therapeutic method suitable for use in methods of the invention (i.e., administration of a cysteamine compound) for the treatment of and/or prevention of the development of allergies as well as the symptoms associated with an allergic reaction (i.e., antihistamines, corticosteroids). For example, a compound can be provided in admixture with a cysteamine compound, such as in a pharmaceutical composition; or the compound and cysteamine can be provided as separate compounds, such as, for example, separate pharmaceutical compositions administered consecutively, simultaneously, or at different times. Preferably, if the cysteamine compound and the known agent (or therapeutic method) are administered separately, they are not administered so distant in time from each other that the cysteamine compound and the known agent cannot interact.

As used herein, reference to a “cysteamine compound” includes cysteamine, the various cysteamine salts (such as cysteamine hydrochloride and cysteamine phosphate) as well as prodrugs of cysteamine that can, for example, be readily metabolized in the body to produce cysteamine. Also included within the scope of the subject invention are analogs, derivatives, conjugates, and metabolites of cysteamine, which have the ability as, described herein to treat and/or prevent the development of allergies as well as the symptoms associated with an allergic reaction. Various analogs, derivatives, conjugates, and metabolites of cysteamine are well known and readily used by those skilled in the art and include, for example, compounds, compositions and methods of delivery as set forth in U.S. Pat. Nos. 6,521,266; 6,468,522; and 5,714,519.

As contemplated herein, a cysteamine compound includes pantothenic acid. Pantothenic acid is a naturally occurring vitamin that is converted in mammals to coenzyme A, a substance vital to many physiological reactions. Cysteamine is a component of coenzyme A, and increasing coenzyme A levels results in increased levels of circulating cysteamine. Alkali metal salts, such as magnesium phosphate tribasic and magnesium sulphite (Epsom salts), enhance formation of coenzyme A. Furthermore, breakdown of coenzyme A to cysteamine is enhanced by the presence of a reducing agent, such as citric acid. Thus, the combination of pantothenic acid and alkali metal salts results in increased coenzyme A production and, concomitantly, cysteamine.

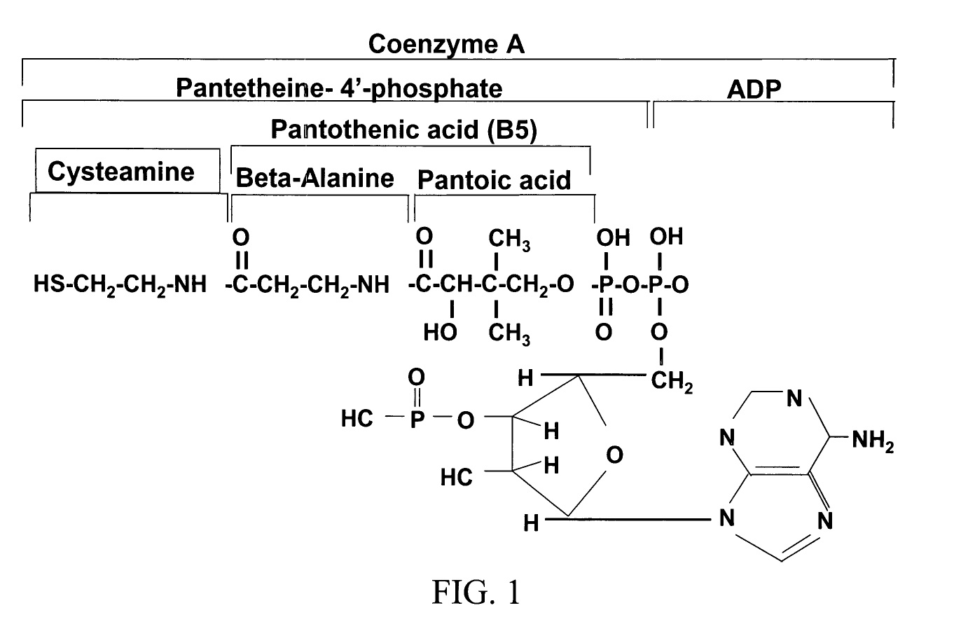

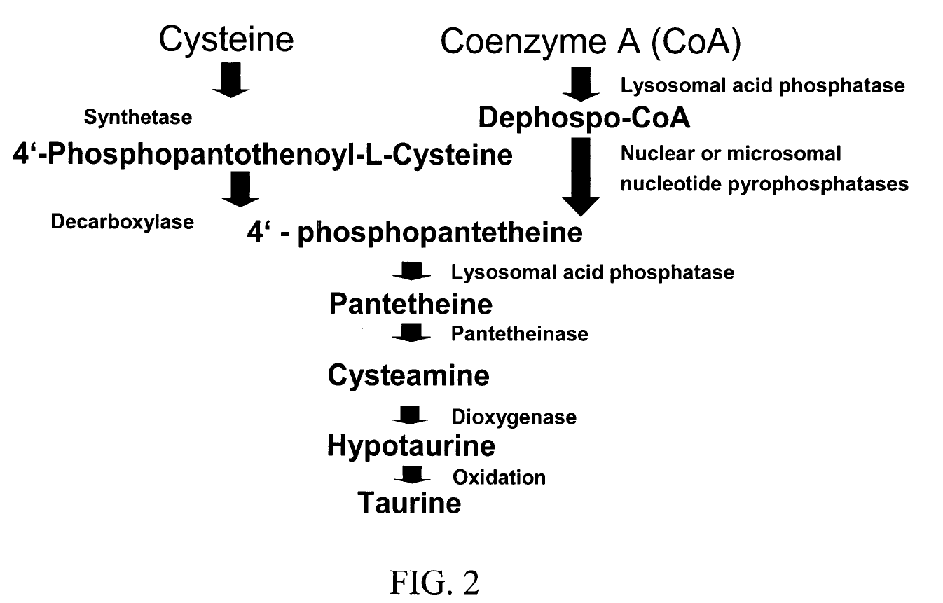

Accordingly, in one embodiment of the subject invention, the advantages of cysteamine, as set forth herein, can be achieved by promoting the endogenous production of cysteamine through natural metabolic process such as through the action of co-enzyme A or as a metabolite of cysteine (see FIGS. 1 and 2). This can be achieved by, for example, the administration of pantothenic acid.

The term “effective amount,” as used herein, refers to the amount necessary to elicit the desired biological response. In accordance with the subject invention, the effective amount of cysteamine is the amount necessary to decrease a particular sign and/or symptom (i.e., rhinorrhea, watery eyes, purities, drop in blood pressure, drop in body temperature, level of IgE, etc.) of an allergic reaction. The decrease may be a 10%, 20%, 30%, 40%, 50%, 60%, 70%, 80%, 90%, 95%, 98% or 99% decrease. The effective amount of cysteamine in a tolerizing composition is the amount that, when administered to a sensitized patient, results in tolerization of the patient to the antigen.

Specifically exemplified herein is the use of cysteamine hydrochloride (and/or analogs, derivatives and prodrugs thereof) to treat and/or prevent the onset of symptoms in an allergic and/or sensitized patient; or to reduce the severity, intensity, or duration of subsequently developed symptoms. Undesirable and unpleasant symptoms associated with allergic reactions such as, and not limited to, airway hyperresponsiveness, hives, rash, purities, watery eyes, runny nose, bronchoconstriction, edema, diarrhea, vasodilation, headache, decreased blood pressure, coughing, wheezing, and asthma, can be treated, prevented, and/or reduced through consumption, according to the subject invention, of a cysteamine compound.

Further advantages of the subject invention include treatment and/or prevention of the development of allergies. Specifically, a cysteamine compound (and/or analogs, derivatives, and prodrugs thereof) can be administered prior to or with exposure to an antigen to cause the immune system to adopt a less overactive response. Alternatively, a cysteamine compound can be administered concurrently with other known agents and/or therapies used to treat and/or prevent the development of allergies as well as the symptoms associated with an allergic reaction.

In accordance with the subject invention, a cysteamine compound can be administered concurrently with common allergy-related medications including, without limitations, antihistamines (i.e., terfenadine, astemazole, loratadine); decongestants (i.e., pseudoephedrine); steroids (i.e., beclomethasone, triamcinolone, budesonide, fluticasone); non-steroidal anti-inflammatory medications (i.e., cromolyn sodium, nedocromil); epinephrine; and bronchodilators (i.e., beta-agonists, anticholinergics).

A cysteamine compound, according to the subject invention, can also be administered concurrently with therapeutic methods associated with the treatment and/or prevention of allergies and/or symptoms associated with an allergic reaction. Such therapeutic methods include, and are not limited to, gene therapies and immunotherapties (i.e., allergy shots used to dull the immune response to allergens).

The compositions of the invention can be used in a variety of routes of administration, including, for example, orally-administrable forms such as tablets, capsules or the like, or via parenteral, intravenous, intramuscular, transdermal, buccal, subcutaneous, suppository, or other route. Such compositions are referred to herein generically as “pharmaceutical compositions.” Typically, they can be in unit dosage form, namely, in physically discrete units suitable as unitary dosages for human consumption, each unit containing a predetermined quantity of active ingredient calculated to produce the desired therapeutic effect in association with one or more pharmaceutically acceptable other ingredients, i.e., diluent or carrier.

The cysteamine compounds of the subject invention can be formulated according to known methods for preparing pharmaceutically useful compositions. Formulations are described in a number of sources, which are well known and readily available to those skilled in the art. For example, Remington's Pharmaceutical Science (Martin E W [1995] Easton Pa., Mack Publishing Company, 19^{th} ed.) describes formulations that can be used in connection with the subject invention. Formulations suitable for parenteral administration include, for example, aqueous sterile injection solutions, which may contain antioxidants, buffers, bacteriostats, and solutes, which render the formulation isotonic with the blood of the intended recipient; and aqueous and nonaqueous sterile suspensions, which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

Administration of the cysteamine, in accordance with the subject invention, can be accomplished by any suitable method and technique presently or prospectively known to those skilled in the art. In a preferred embodiment, the cysteamine compound is formulated in a patentable and easily consumed oral formulation such as a pill, lozenge, tablet, gum, beverage, etc. The consumption is then taken at, shortly before, or after, the time of introduction to an antigen.

In accordance with the invention, compositions comprising, as an active ingredient, an effective amount of the cysteamine compound and one or more non-toxic, pharmaceutically acceptable carrier or diluent. Examples of such carriers for use in the invention include ethanol, dimethyl sulfoxide, glycerol, silica, alumina, starch, sorbitol, inosital, xylitol, D-xylose, manniol, powdered cellulose, microcrystalline cellulose, talc, colloidal silicon dioxide, calcium carbonate, magnesium cabonate, calcium phosphate, calcium aluminium silicate, aluminium hydroxide, sodium starch phosphate, lecithin, and equivalent carriers and diluents.

To provide for the administration of such dosages for the desired therapeutic treatment, compositions of the invention will typically comprise between about 0.1% and 45%, of the total composition including carrier or diluent. The dosage used can be varied based upon the age, weight, health, or the gender of the individual to be treated.

In one embodiment, the dosage of cysteamine administered to a patient to elicit a desired response is substantially 500 mg or greater. The desired response can include (1) a reduction in the severity, duration, or intensity of symptoms related to an allergic reaction; (2) decrease or elimination in immune response, including decrease and/or elimination of IgE response; (3) elimination of symptoms associated with an allergic reaction; and (4) tolerization of a patient to an antigen. In a preferred embodiment, the dosage of cysteamine administered to a patient to elicit a desired response is about 500-700 mg. More preferably, the dosage of cysteamine administered to a patient to elicit a desired response is about 600 mg.

All patents, patent applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for treating allergies wherein said method comprises administering to a patient an effective amount of a cysteamine compound.

2. The method, according to claim 1, wherein said cysteamine compound is selected from the group consisting of cysteamine, cysteamine salts, prodrugs of cysteamine, analogs of cysteamine, derivatives of cysteamine, conjugates of cysteamine, and metabolites of cysteamine.

3. The method, according to claim 2, wherein said cysteamine salt is cysteamine hydrochloride or cysteamine phosphate.

4. The method, according to claim 1, wherein said patient is an allergic patient.

5. The method, according to claim 1, wherein said patient is a sensitized patient.

6. The method, according to claim 1, wherein said cysteamine compound is taken orally, parenterally, intravenously, intramuscularly, transdermally, via buccal route, subcutaneously, or via suppository.

8. A method for tolerizing a sensitized patient to an antigen, wherein said method comprises administering to said patient an effective amount of a cysteamine compound.

9. The method, according to claim 8, wherein said cysteamine compound is selected from the group consisting of cysteamine, cysteamine salts, prodrugs of cysteamine, analogs of cysteamine, derivatives of cysteamine, conjugates of cysteamine, and metabolites of cysteamine.

10. The method, according to claim 9, wherein said cysteamine salt is cysteamine hydrochloride or cysteamine phosphate.

11. The method, according to claim 8, wherein said cysteamine compound is taken orally, parenterally, intravenously, intramuscularly, transdermally, via buccal route, subcutaneously, or via suppository.

12. A method for reducing the severity, intensity, or duration of symptoms associated with an allergic reaction, wherein said method comprises administering to a patient an effective amount of a cysteamine compound.

13. The method, according to claim 12, wherein said cysteamine compound is selected from the group consisting of cysteamine, cysteamine salts, prodrugs of cysteamine, analogs of cysteamine, derivatives of cysteamine, conjugates of cysteamine, and metabolites of cysteamine.

14. The method, according to claim 13, wherein said cysteamine compound is cysteamine hydrochloride and cysteamine phosphate.

15. The method, according to claim 12, wherein said patient is an allergic patient or a sensitized patient.

16. The method, according to claim 12, wherein said cysteamine compound is taken orally, parenterally, intravenously, intramuscularly, transdermally, via buccal route, subcutaneously, or via suppository.

17. A method for preventing the onset of symptoms associated with an allergic reaction, wherein said method comprises administering to a patient an effective amount of a cysteamine compound.

18. The method, according to claim 17, wherein said cysteamine compound is selected from the group consisting of cysteamine, cysteamine salts, prodrugs of cysteamine, analogs of cysteamine, derivatives of cysteamine, conjugates of cysteamine, and metabolites of cysteamine.

19. The method, according to claim 18, wherein said cysteamine compound is selected from the group consisting of cysteamine hydrochloride and cysteamine phosphate.

20. The method, according to claim 17, wherein said patient is an allergic patient or a sensitized patient.

21. The method, according to claim 17, wherein said cysteamine compound is taken orally, parenterally, intravenously, intramuscularly, transdermally, via buccal route, subcutaneously, or via suppository.

22. A method for preventing the development of allergies, wherein said method comprises administering to a patient an effective amount of a cysteamine compound.

23. The method, according to claim 22, wherein said cysteamine compound is selected from the group consisting of cysteamine, cysteamine salts, prodrugs of cysteamine, analogs of cysteamine, derivatives of cysteamine, conjugates of cysteamine, and metabolites of cysteamine.

24. The method, according to claim 23, wherein said cysteamine compound is cysteamine hydrochloride and cysteamine phosphate.

25. The method, according to claim 22, wherein said patient is an allergic patient or a sensitized patient.

26. The method, according to claim 22, wherein said cysteamine compound is taken orally, parenterally, intravenously, intramuscularly, transdermally, via buccal route, subcutaneously, or via suppository.
